(19) United States
(12) Patent Application Publication
(10) Pub. No.: US 2005/0154375 A1
(43) Pub. Date: Jul. 14, 2005

(54) Surgical instrument having a rotational ring

(75) Inventors: Michael J. McGowan SR., Barrington, NJ (US); Charles R. Hurst JR., Bullard, TX (US); Harry Michael Lambert, Houston, TX (US); Christopher F. Lumpkin, Evergreen, CO (US)

Correspondence Address: SYNNESTVEDT & LECHNER, LLP, 2600 ARAMARK TOWER, 1101 MARKET STREET, PHILADELPHIA, PA, 191072950, US

(73) Assignee: Innovatech Surgical, Inc., Camden, NJ (US)

(21) Appl. No.: 10/899,628

(22) Filed: Jul. 27, 2004

Related U.S. Application Data
(60) Provisional application No. 60/490,567, filed on Jul. 28, 2003.

(51) Int. Cl. A61B 17/00

(52) U.S. Cl. 606/1

(57) ABSTRACT

A surgical instrument having a rotational ring is described. In one implementation, the surgical instrument includes a handle and a rotational ring. The handle includes at least one actuator member configured to operate a surgical tool located at a tip of the surgical instrument by moving the actuator member. The rotational ring is positioned in substantially close proximity to the one or more actuator members. Rotation of the rotational ring causes the surgical tool located at the tip of the surgical instrument to rotate in a corresponding rotational fashion.

### CROSS-REFERENCE TO RELATED APPLICATIONS

The present patent application claims benefit of U.S. Provisional Application Ser. No. 60/490,567 filed on Jul. 28, 2003. The content of the aforementioned application is fully incorporated by reference herein.

### TECHNICAL FIELD

The present invention relates generally to a handheld microsurgical surgical instrument.

### BACKGROUND

Microsurgical surgical instruments are typically handheld instruments used to perform surgical procedures on delicate parts of human or animal anatomies. For example, microsurgical instruments are commonly used for performing eye surgery. Such instruments include a handle portion to hold the surgical instrument and a tip designed for insertion inside an incision made in the eye. The tips typically include some type of micro-surgical tool, such as a scalpel, scissors, forceps, suction device, or other related surgical tools. Many microsurgical instruments also typically include one or more actuating levers to operate the surgical tool at the tip of the surgical instrument. Pressing or depressing the actuating levers on the shaft of the microsurgical instrument, causes the surgical tool to operate. Additionally, some microsurgical instruments may include some rotational mechanism configured to permit the surgeon or an assistant in rotating the surgical tool located at the tip of the instrument when maneuvering the surgical instrument.

When performing surgery, such as vitreoretinal surgery (i.e., back of the eye surgery), three small incisions may be made in the eye to gain access to the back of the eye. In a first incision, an infusion cannula is inserted into the eye to maintain an infusion line for inflating the eye so it does not collapse during surgery and/or flushing/draining the eye after surgery, etc. In a second incision, the surgeon typically inserts some type of illuminating device or magnifying scope to observe the operation inside the eye. And in a third incision, a surgeon typically inserts the tip of the microsurgical instrument to perform some type of operation or manipulation. The surgeon views such surgery almost exclusively through a microscope such as a stereoscopic microscope, or other related magnifying device.

Accordingly, while viewing the surgery through some type of scope, the surgeon often relies blindly, on touch and feel, to manipulate and operate the microsurgical instrument. Unfortunately, despite attempts to improve the operational characteristics and refine the feel of microsurgical instruments, most remain cumbersome and difficult to manipulate, especially when viewing the operation through some type of scope.

For instance, many surgeons find it necessary to use two hands, either the surgeon's second hand or an assistant's hand, to steady the instrument when rotating the surgical tool in conjunction with manipulating the surgical tip. Using two hands on a microsurgical instrument, however, is awkward and further increases the chances of inaccurate movements, exaggerated movement, poor coordination, accidental slippage of the instrument, and other awkward situations, which may increase the potential risk of damaging the eye or other body part.

### SUMMARY

A surgical instrument having a rotational ring is described. In one implementation, the surgical instrument includes a handle and a rotational ring. The handle includes at least one actuator member configured to operate a surgical tool located at a tip of the surgical instrument by moving the actuator member. The rotational ring is positioned in substantially close proximity to the one or more actuator members. Rotation of the rotational ring causes the surgical tool located at the tip of the surgical instrument to rotate in a corresponding circular fashion.

The following description, therefore, introduces the concept of providing a rotational ring in substantially close proximity to one or more actuator members to enable a surgeon to use one hand to manipulate the rotational ring in conjunction with the one or more actuator members while performing a surgical procedure. The close proximity of the rotational ring to the one or more actuator members also permits a user of the surgical instrument to use the same hand to manipulate simultaneously the rotational ring in conjunction with the one or more actuator members while performing a surgical procedure without having to look at the surgical instrument, use two hands, and/or receive assistance from others to hold, steady or manipulate the surgical instrument.

Additional features, such as gripping elements included on the rotational ring, further permit the surgeon to locate, feel, and rotate the rotational ring blindly, without having to see the surgical instrument, use two hands, or rely on an assistant to hold or manipulate the instrument.

Further features include a surface pattern, such as knurling on the one or more of the actuation members of the surgical instrument for providing a high coefficient of friction between the handle and a surgeon's hand when holding and handling the instrument. For instance, a knurled pattern provides a secure coefficient of friction between the surgeon's hand and the actuation members when the surgical instrument is exposed to fluid such as saline, blood, silicone oil, and so forth.

### BRIEF DESCRIPTION OF THE DRAWINGS

The detailed description is described with reference to the accompanying figures. In the figures, the left-most digit(s) of a reference number identifies the figure in which the reference number first appears. It should be noted that the figures are not necessarily drawn to scale and are for illustration purposes only.

FIG. 1 illustrates a side planar view of a surgical instrument.

FIG. 2 shows a cross sectional view of a rotational ring.

FIG. 3 illustrates a side view of a surgical instrument with a “Sutherland” style handle.

### DETAILED DESCRIPTION

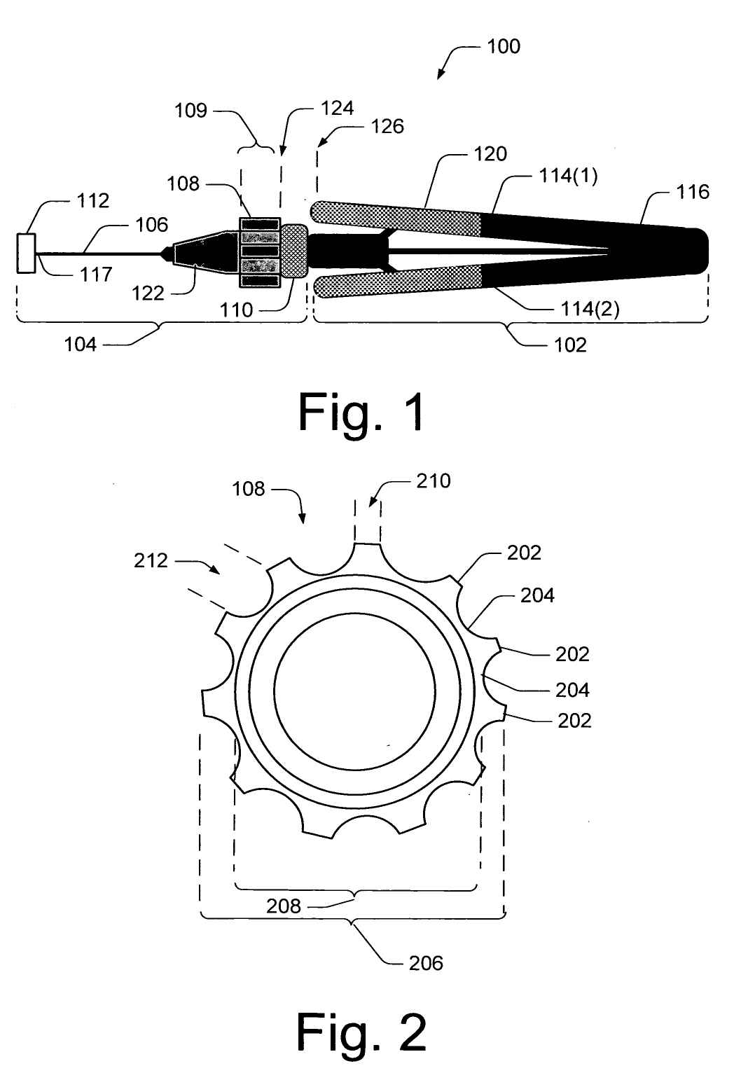

FIG. 1 illustrates a side view of a surgical instrument 100. Surgical instrument 100 includes a handle 102, a tool tip assembly 104, a cannula 106, a rotational ring 108, a tool tip assembly connector 110, and a surgical tool 112. Surgical instrument 100 is typically used in microsurgical applications such as vitreoretinal surgery; however, surgical instrument 100 may be adapted for use on other anatomical structures and for other surgical procedures.

In the exemplary illustration, handle 102 is commonly referred to as a “squeeze handle” surgical instrument including actuation members 114(1) and 114(2). Actuation members 114(1) and 114(2) are joined at a base 116 of handle 102 and are held apart by internal mechanisms such as a spring (not shown). When actuation members 114(1) and 114(2) are squeezed, the compression action causes an internal piston (not shown) to move causing activation, or alternatively deactivation, of surgical tool 112. Correspondingly, when actuation members 114(1) and 114(2) are released to an uncompressed state as shown in FIG. 1, the decompression action causes the internal piston to move causing activation, or alternatively deactivation, of a particular surgical tool 112.

It is appreciated that the internal parts used in squeeze handle surgical instruments are well known by those skilled in the art. For example, for more information illustrating basic functionality and various internal components of such a handle, please refer to U.S. Pat. No. 5,893,873 to Rader et al., U.S. Pat. No. 5,370,658 to Scheller et al., and/or U.S. Pat. No. 4,955,887 to Zirm all incorporated herein by reference in their entirety. It is also appreciated that handle 102 may only include one actuation member 114(1) or 114(2), instead of two actuation members 114(1) and 114(2) or a plurality of actuation members in excess of two.

Each actuation member referred to generally as reference number 114 may have a rounded surface substantially concentric with a center axis of handle 102. The rounded surface may include a knurled pattern 120, which provides a high coefficient of friction between the handle and a surgeon's hand (or assistant's hand) when holding the instrument 100. Knurled pattern 120 also provides a secure coefficient of friction between the surgeon's hand and the handle 102 when instrument 100 is exposed to fluid such as saline, blood, silicone oil, and so forth.

Tool tip assembly 104 is detachably coupled to handle 102 via a tool tip assembly connector 110. In the exemplary implementation, tool tip assembly connector 110 is a threaded female connector that fits over a male threaded end (not shown) of handle 102. Alternatively, tool tip assembly 104 may be integrally connected to handle 102, or may be attached by other mechanisms such as snap-on connectors, clips, or other attachment/detachment coupler mechanisms.

It is appreciated that the internal parts (not shown) used in tool tip assembly 104 are well known by those skilled in the art. For example, further details of a Sutherland style rotational sleeve (tool tip assembly) are described in U.S. Pat. No. 4,258,716 to Sutherland (hereinafter the '716 Patent), which is hereby incorporated herein by reference in its entirety.

Located at the distal end 117 of tool tip assembly 104 is a surgical tool 112 represented as a single block for purposes of illustration. It is noted that block 112 may represent any number of various surgical tools, such as, but not limited to, forceps, scissors, scalpels, clamps, and so forth. Typically, operation of any one of these surgical tools can be accomplished by moving the one or more actuating members 114. As used herein, “operating” the surgical tool means opening, closing, moving, activating, deactivating, clamping, and/or manipulating the surgical tool 112. For example, squeezing actuating members 114 may cause a surgical tool 112 such as forceps to close, whereas releasing actuating members 114 may cause the forceps to open.

Cannula 106 is attached to a cone shaped piece 122 and is of an applicable size, such as a 20-gauge or 25-gauge cannula, for insertion into the eye. Cone shaped piece 122 includes rotational ring 108. It should be noted that cone shaped piece 122 may not be used and cannula 106 may be attached directly to rotational ring 108. Alternatively, cone shaped piece 122 may be implemented in other shapes such as a cylindrical shape, square, and so forth. In the exemplary implementation, cannula 106, cone shaped piece 122, and rotational ring 108 are integrally connected, although it is appreciated that these components may be separate and move independently from each other.

Rotational ring 108 is configured to rotate surgical tool 112 located at cannula 106 by correspondingly rotating, i.e., turning the rotation ring 108 in a clockwise or counter clockwise direction. Since rotational ring 108 is integrally connected to surgical tool 112 via cannula 106 and cone shaped piece 122, the rotational motion of rotational ring 108 is translated to surgical tool 112.

Rotational ring 108 includes an outer edge 124 proximal to actuator member(s) 114 located on handle 102. Accordingly, rotation ring 108 is positioned in substantially close proximity to the one or more actuator member(s) 114. As used herein “substantially close proximity” means a distance between the outer edge 124 of rotational ring 108 and the actuator member(s) 114, which permits an average-sized woman's hand to simultaneously grip and move actuator member(s) 114 while comfortably rotating the rotational ring 108 with the same hand. For instance in one exemplary implementation, rotational ring 108 is positioned no further than about 0.300 inches from the actuator member 114 measured from a surface 126 on actuator member 114 closest to an outer edge 124 of rotational ring 108 proximal to actuator member 114.

Accordingly, the close proximity of rotational ring 108 to the one or more actuator members 114 permits a user of the surgical instrument to use one hand to simultaneously manipulate rotational ring 108 in conjunction with one or more actuator members 114 while performing a surgical procedure. The close proximity of rotational ring 108 to actuator members 114 also reduces the chance that a surgeon will need to look at the surgical instrument in order to find rotational ring 108. Furthermore, the close proximity of rotational ring 108 to actuator members 114 greatly reduces or eliminates the need for a second hand, to hold, steady and/or manipulate the surgical instrument.

In one exemplary implementation, rotational ring 108 has a width 109 of approximately 0.245 inches, which may provide superior perceptive feel and higher proprioceptive confidence to a surgeon than a narrower width. Nevertheless, it is appreciated that in alternative implementations, width 109 may be wider or slightly narrower than 0.245 inches.

Besides the width 109 of rotational ring 108 and close proximity of the rotational ring 108 to actuation members 114, rotational ring 108 also includes distinguished gripping elements to permit the surgeon to confidently find and feel the rotational ring 108, such as when viewing an operation through some type of scope and relying on blind touch and feel to operate the surgical instrument. For example, FIG. 2 shows a cross sectional view of rotational ring 108. In one exemplary implementation, rotational ring 108 includes gripping elements 202 separated by gaps 204. Gripping elements 202 permit the surgeon to confidently find and feel rotational ring 108. Gripping elements 202 may take various forms, shapes and sizes other than those shown in FIG. 2. However, gripping elements 202 should have a distinct feel so that the surgeon can easily locate and recognize rotational ring 108 by touch when searching for it. Additionally, gripping elements may be separated by gaps 204 to further delineate individual gripping elements 202 and permit the surgeon to precisely rotate rotational ring 108 in a clockwise or counterclockwise direction. In this example, gaps 204 are pronounced indentations permitting the surgeon to readily distinguish and grip gripping elements 202 without slipping, such as when turning rotational ring 108.

In one exemplary implementation, gripping elements 202 have a collective outer circumference 206 measured at a top-most surface of each gripping element which is about 1.351 inches or greater. And in one exemplary implementation, gaps 204 have a collective inner circumference 208 measured at an inner-most surface of each gap 206, which is about 1.099 inches or smaller. In one exemplary implementation, each gripping element 202 has width 210 of approximately 0.058 inches and each gap 204 has a width of approximately 0.091 inches. However, other widths, greater or smaller, could be selected for gripping elements 202 and gaps 204.

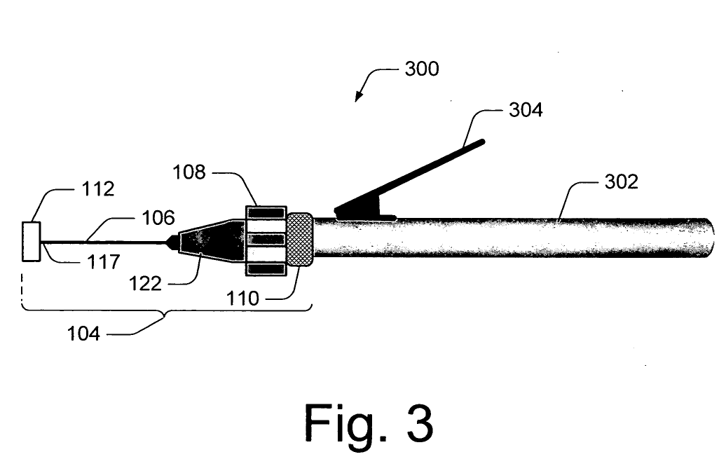

It is noted that tool tip assembly 104 may be attached to other types of handles. For example, FIG. 3 illustrates a side view of a surgical instrument 300 having “Sutherland” style handle 302. For example, further details of a Sutherland style handle are described in the '716 Patent. Surgical instrument 300 includes handle 302 and a single actuation member in the form of a lever 304. Like squeeze handle 102, tool tip assembly 104 may be detachably coupled to handle 302 via tool tip assembly connector 110 by screwing tool tip assembly connector 110 onto a male threaded end (not shown) of handle 302. Alternatively, tool tip assembly 104 may be integrally connected to handle 302, or may be attached by other mechanisms such as snap-on connectors, clips, or other attachment/detachment coupler mechanisms.

Although handle 302 is illustrated as being a cylindrical shape, it is appreciated that handle 302 may also have planar or curved sides similar to a pen or pencil. Additionally, handle 302 may include some type of gripping pattern such as knurled pattern 120 shown in FIG. 1. It is also noted that tool tip assembly 104 and its constituent components may be manufactured and sold as separate apparatuses from either handle 102 or handle 302.

Although FIGS. 1 and 3 show cone shaped piece 122 and rotational ring 108 as being separate and adjacent components, it is appreciated that rotational ring 108 may fit over cone shaped piece 122. For instance rotational ring 108 may be made of a pliable material such as rubber or silicone, designed to surround at least a portion of cone shaped piece 122. Alternatively, rotational ring 108 also may comprise a portion of cone shaped piece 122 or the entire portion of cone shaped piece 122, possibly eliminating cone shaped piece 122 from tool tip assembly 104.

Although the invention has been described in language specific to structural features and/or methodological acts, it is to be understood that the invention defined in the appended claims is not necessarily limited to the specific features or acts described. Rather, the specific features and acts are disclosed as exemplary forms of implementing the claimed invention.

1. A surgical instrument, comprising:
a handle having at least one actuator member configured to operate a surgical tool located at a tip of the surgical instrument by moving the actuator member; and
a rotational ring positioned in substantially close proximity to the actuator member, wherein rotation of the rotational ring causes the surgical tool located at the tip of the surgical instrument to rotate in a corresponding rotational fashion.

2. The surgical instrument as recited in claim 1, wherein the substantially close proximity of the rotational ring to the actuator member permits a user of the surgical instrument to use the same hand to simultaneously manipulate the rotational ring in conjunction with the actuator member while performing a surgical procedure.

3. The surgical instrument as recited in claim 1, wherein the rotational ring is positioned no further than about 0.300 inches from the actuator member measured from a surface on the actuator member closest to an outer edge of the rotational ring proximal to the actuator member.

4. The surgical instrument as recited in claim 1, wherein the rotational ring comprises gripping elements separated by gaps, wherein the gripping elements have a collective outer circumference measured at a top-most surface of each gripping element which is about 1.351 inches or greater and the gaps have a collective inner circumference measured at an inner-most surface of each gap which is about 1.099 inches or smaller.

5. The surgical instrument as recited in claim 1, wherein the rotational ring comprises gripping elements separated by gaps, wherein each of the gaps has a width of about 0.091 inches separating two adjacent gripping elements.

6. The surgical instrument as recited in claim 1, wherein the rotational ring comprises gripping elements that are each about 0.058 inches wide.

7. The surgical instrument as recited in claim 1, wherein the actuator member comprises a knurled pattern.

8. A surgical instrument, comprising:
a handle comprising one or more actuator members configured to operate a surgical tool located at a tip of the surgical instrument by moving at least one of the actuator members; and
a rotational ring positioned in substantially close proximity to the actuator member(s), the rotational ring comprising gripping elements, each gripping element separated by a gap large enough to enable a surgeon to easily recognize the rotational ring by touch, the gripping elements also being large enough to enable the surgeon to maintain contact with the rotational ring without slipping when rotating the rotational ring during surgery, wherein the rotation of the rotational ring causes the surgical tool located at the tip of the surgical instrument to rotate in a corresponding rotational fashion.

9. The surgical instrument as recited in claim 8, wherein the substantially close proximity is a distance measured from a surface on the actuator member(s) to an outer edge of the rotational ring proximal to the actuator member, which permits an average-sized woman's hand to simultaneously grip and move the actuator member(s) while comfortably rotating the rotational ring with the same hand.

10. The surgical instrument as recited in claim 8, wherein the rotational ring is positioned no further than about 0.300 inches from the actuator member(s) measured from a surface, on the actuator member(s), closest to an outer edge of the rotational ring proximal to the actuator member(s).

11. The surgical instrument as recited in claim 8, wherein the gripping elements have a collective outer circumference measured at a top-most surface of each gripping element which is about 1.351 inches or greater and the gaps have a collective inner circumference measured at an inner-most surface of each gap which is about 1.099 inches or smaller.

12. The surgical instrument as recited in claim 8, wherein each of the gaps has a width of about 0.091 inches separating two adjacent gripping elements.

13. The surgical instrument as recited in claim 8, wherein the gripping elements are each about 0.058 inches wide.

14. The surgical instrument as recited in claim 8, wherein the actuator member(s) comprises a knurled pattern.

15. A surgical instrument, comprising:
one or more actuator members for operating a surgical tool located at a tip of the surgical instrument by moving the actuator member(s); and
a rotational ring positioned no further than about 0.300 inches from the actuator member(s) measured from a surface, on the actuator member(s), closest to an outer edge of the rotational ring proximal to the actuator member(s).

16. The surgical instrument as recited in claim 15, wherein the rotational ring comprises gripping elements separated by gaps, wherein the gripping elements have a collective outer circumference measured at a top-most surface of each gripping element which is about 1.351 inches or greater and the gaps have a collective inner circumference measured at an inner-most surface of each gap which is about 1.099 inches or smaller.

17. The surgical instrument as recited in claim 15, wherein the rotational ring comprises gripping elements separated by gaps, wherein each of the gaps has a width of about 0.091 inches separating two adjacent gripping elements.
